(19) United States
(12) Patent Application Publication
(10) Pub. No.: US 2023/0157885 A1
(43) Pub. Date: May 25, 2023

(54) OPHTHALMIC SURGICAL SYSTEM WITH A DMD CONFOCAL MICROSCOPE

(71) Applicant: Alcon Inc., Fribourg (CH)

(72) Inventors: Edward A. DeHoog, Long Beach, CA (US); John Park, Irvine, CA (US)

(21) Appl. No.: 18/054,448

(22) Filed: Nov. 10, 2022

Related U.S. Application Data
(60) Provisional application No. 63/281,422, filed on Nov. 19, 2021.

(51) Int. Cl. A61F 9/008

(52) U.S. Cl. CPC A61F 9/00825; A61F 2009/00897; A61F 2009/00874; A61F 2009/00851

(57) ABSTRACT

In certain embodiments, an ophthalmic laser surgical system for imaging and treating a target in an eye includes a digital micromirror device (DMD) confocal microscope, a laser device, and a computer. The DMD confocal microscope generates of images of the eye and includes a light source, a DMD device, and an image sensor. The light source provides a microscope imaging beam. The DMD device directs the microscope imaging beam along an imaging path towards the eye, receives the microscope imaging beam reflected from the eye, and rejects light of the reflected microscope imaging beam that is not from an image plane to scan the microscope imaging beam. The image sensor detects the scanned microscope imaging beam to generate the images of the eye. The laser device directs a laser beam along a laser beam path towards the target in the eye.

### TECHNICAL FIELD

The present disclosure relates generally to ophthalmic surgical systems, and more particularly to ophthalmic surgical systems with a digital micromirror device (DMD) confocal microscope.

### BACKGROUND

Eye floaters are clumps of collagen in the vitreous that can degrade vision quality, e.g., visual acuity and contrast sensitively. Laser vitreolysis removes floaters by directing a laser beam at a floater to disintegrate the floater. However, known laser vitreolysis systems yield poor image resolution of floaters and fail to provide depth location of floaters, which makes accurately targeting floaters difficult. Moreover, if a floater is too close to the retina, the lack of depth location may result in retinal damage.

### BRIEF SUMMARY

In certain embodiments, an ophthalmic laser surgical system for imaging and treating a target in an eye includes a digital micromirror device (DMD) confocal microscope, a laser device, and a computer. The DMD confocal microscope generates of images of the eye. An axis of the eye defines a z-axis, and the z-axis defines xy-planes. The DMD confocal microscope includes a light source, a DMD device, and an image sensor. The light source provides a microscope imaging beam. The DMD device directs the microscope imaging beam along an imaging path towards the eye, receives the microscope imaging beam reflected from the eye, and rejects light of the reflected microscope imaging beam that is not from an image plane to scan the microscope imaging beam. The image sensor detects the scanned microscope imaging beam to generate the images of the eye. The laser device directs a laser beam along a laser beam path towards the target in the eye. The computer sends instructions to the DMD confocal microscope and the laser device.

Embodiments may include none, one, some, or all of the following features:

The target comprising a vitreous eye floater.

The DMD confocal microscope generates two-dimensional (2D) enface images at the xy-planes. The DMD confocal microscope may combine the two-dimensional (2D) enface images to generate a three-dimensional (3D) image.

The DMD device rejects light of the reflected microscope imaging beam that is not from an image plane to scan the microscope imaging beam by toggling on a set of micromirror(s) that operate as one or more pinhole(s) to scan the microscope imaging beam.

The ophthalmic laser surgical system includes an imaging system that generates additional images of the eye. The ophthalmic laser surgical system may include an optical coherence tomography (OCT) device and/or a scanning laser ophthalmoscope (SLO) device that generates the additional images. The imaging system may determine a z-location of the target relative to the z-axis. The ophthalmic laser surgical system includes an xy-scanner that: receives an imaging beam from the imaging system and directs the imaging beam along an imaging system beam path towards the eye; and receives the laser beam from the laser device and directs the laser beam along the laser beam path aligned with the imaging system beam path towards the eye.

In certain embodiments, a method for imaging and treating a target in an eye comprises generating, by a digital micromirror device (DMD) confocal microscope, images of the eye. An axis of the eye defines a z-axis, which in turn defines xy-planes. The generating comprises: providing, by a light source of the DMD confocal microscope, a microscope imaging beam; directing, by an array of micromirrors of a DMD device of the DMD confocal microscope, the microscope imaging beam along an imaging path towards the eye; receiving, by the array of micromirrors, the microscope imaging beam reflected from the eye; rejecting, by the array of micromirrors, light of the microscope imaging beam reflected from the eye that is not from an image plane to scan the microscope imaging beam; and detecting, by an image sensor of the DMD confocal microscope, the scanned microscope imaging beam to generate the images of the eye. A laser beam is directed by a laser device along a laser beam path towards the target in the eye.

Embodiments may include none, one, some, or all of the following features:

The target comprises a vitreous eye floater.

Rejecting light of the microscope imaging beam reflected from the eye that is not from the image plane to scan the microscope imaging beam comprises toggling on a set of one or more micromirrors that operate as a pinhole to scan the microscope imaging beam.

Rejecting light of the microscope imaging beam reflected from the eye that is not from the image plane to scan the microscope imaging beam comprises toggling on a set of one or more micromirrors that operate as multiple pinholes to scan the microscope imaging beam.

Two-dimensional (2D) enface images are generated by the DMD confocal microscope at the xy-planes. The two-dimensional (2D) enface images may be combined by the DMD confocal microscope to generate a three-dimensional (3D) image.

A second set of images of the eye are generated by an imaging system. At least one of the second images may be generated by an optical coherence tomography (OCT) device or by a scanning laser ophthalmoscope (SLO) device of the imaging system. A z-location of the target relative to the z-axis may be determined by the imaging system. An imaging beam from the imaging system may be received by an xy-scanner and directed along an imaging system beam path towards the eye, and the laser beam from the laser device may be received by the xy-scanner and directed along the laser beam path aligned with the imaging system beam path towards the eye.

### BRIEF DESCRIPTION OF THE DRAWINGS

FIG. 1 illustrates an example of an ophthalmic surgical system that may be used to image and treat a target (such as an eye floater) in an eye, according to certain embodiments;

FIG. 2 illustrates an example of a DMD confocal microscope that may be used in the system of FIG. 1; and

FIG. 3 illustrates an example of a method for imaging and treating an eye that may be performed by an ophthalmic surgical system, such as the system of FIG. 1, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Known laser vitreolysis systems fail to provide accurate and precise imaging and targeting of floaters in certain situations. Accordingly, the ophthalmic surgical systems described herein address these problems. The surgical systems include a digital micromirror device (DMD)-based confocal microscope (“DMD confocal microscope”). The DMD confocal microscope utilizes a DMD as a scanning element, which allows for fast (e.g., greater than 60 hertz (Hz)) image acquisition. The DMD confocal microscope images can be used to determine the xy-location of a floater. The surgical systems may also include an optical coherence tomography (OCT) device that provides the z-location of the floater. Furthermore, the laser treatment and imaging devices may share an xy-scanner, allowing for co-registration among the devices. Thus, these ophthalmic surgical systems provide improved imaging and targeting of floaters.

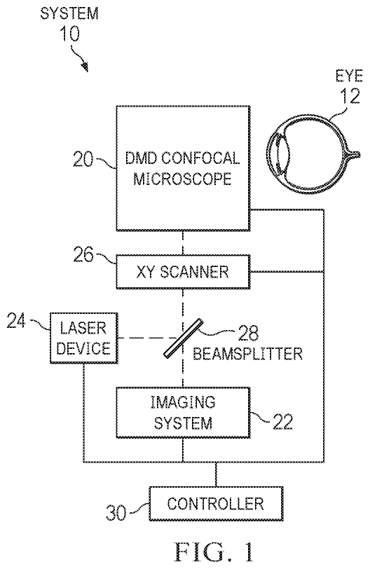

FIG. 1 illustrates an example of an ophthalmic surgical system 10 that may be used to image and treat a target (such as an eye floater) in an eye 12, according to certain embodiments. In the example, system 10 includes a digital micromirror device (DMD)-based confocal microscope (“DMD confocal microscope”) 20, an imaging system 22, a laser device 24, an xy-scanner 26, a beamsplitter 28, and a controller 30, coupled as shown. In the example, an axis (e.g., visual or optical) of eye 12 defines a z-axis, which in turn defines x- and y-axes orthogonal to the z-axis. X- and y-axes define xy-planes within the eye. X-, y-, and z-directions and locations are relative to the x-, y-, and z-axes, respectively.

According to an overview, DMD confocal microscope 20 generates two-dimensional (2D) enface images of the vitreous and retina of eye 12. In certain embodiments, the DMD device has DMD micromirrors that operate extremely quickly and can gather multiple images in parallel. The images may show a shadow that a floater casts onto the retina, which allows for determination of the xy-location of the floater. Imaging system 22 includes an imaging device (e.g., an optical coherent tomography (OCT) device) that can determine the z-location of the floater. Xy-scanner 26 co-registers imaging system 22 and laser device 24 in an xy-plane. Accordingly, system 10 can provide laser device 24 with an accurate xyz-location of the floater, so the laser can fragment the floater. In certain embodiments, controller 30 sends instructions to components of system 10 to perform these operations.

Turning to the components, DMD confocal microscope 20 generates images of eye 12. As an overview, DMD confocal microscope 20 includes a light source, a DMD device, and an image sensor. The light source provides a microscope imaging beam. The DMD device directs the microscope imaging beam along an imaging path towards the eye, receives the microscope imaging beam reflected from the eye, and rejects light of the reflected microscope imaging beam that is not from an image plane to scan the microscope imaging beam. The image sensor detects the scanned microscope imaging beam to generate the images of the eye.

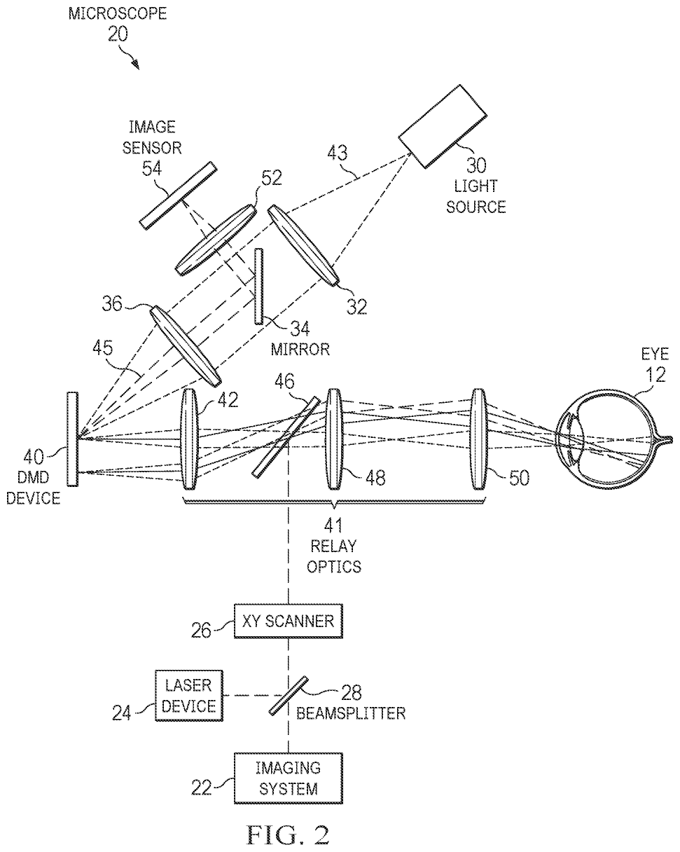

In certain embodiments, the DMD device has DMD micromirrors that operate extremely quickly and can gather multiple images in parallel, so DMD confocal microscope 20 yields faster image acquisition than conventional scanning laser ophthalmoscope (SLO) device or optical coherence tomography (OCT) systems, which use scan optics that provide single image point acquisition. In the embodiments, the DMD is an array of micromirrors that can be toggled to an on or off state that allows light from an image to be transmitted or reflected on a pixel-by-pixel basis. Each micromirror effectively acts as a pinhole in a confocal microscope allowing light only from the object plane to be passed to the image sensor. Out-of-focus light, i.e., light not in the object plane, is rejected i.e., not passed along to the image plane. Micromirrors can be toggled individually in a sequential matter, allowing for the object to be imaged point-by-point, similar to a scanning system. Moreover, by activating multiple pixels in parallel, image acquisition speed can be increased while maintaining confocal sectioning imaging system properties. DMD confocal microscope 20 is described in more detail with reference to FIG. 2.

Imaging system 22 generates images of eye 12 and may include any suitable imaging devices. In certain embodiments, imaging system 22 may include an optical coherence tomography (OCT) device and/or a scanning laser ophthalmoscope (SLO) device. The OCT device may be any suitable interferometer device, e.g., a time domain, spectral domain, and/or swept source OCT device. In certain embodiments, the OCT device may determine a z-location (depth location) of the target relative to the z-axis. For example, the OCT device may generate three-dimensional (3D) images that show the z-location of the target. In certain embodiments, the OCT device may have a depth resolution, such as approximately 5 um or better, to provide an accurate z-location.

Laser device 24 generates a directs a laser beam towards eye 12. The laser beam may have any suitable characteristics to interact with (e.g., photodisrupt, fragment, or photoablate) tissue to treat the target. The laser beam may have any suitable wavelength, e.g., in an ultraviolet or infrared range. Laser device 22 delivers laser pulses of any suitable pulse duration (e.g., in the picosecond to nanosecond range) at any suitable repetition rate (e.g., 30 kilohertz (kHz) or greater).

Xy scanner 26 receives an imaging beam from imaging system 22 and transversely directs the imaging beam along an imaging system beam path towards the eye, and receives a laser beam from laser device 24 and transversely directs the laser beam along the laser beam path aligned with the imaging system beam path towards eye 12. Accordingly, xy scanner 26 co-registers laser device 24 with imaging devices of imaging system 22 for treatment of a floater. Once a floater is located with the imaging devices, the laser can fragment the floater.

Xy-scanner 26 may transversely direct the beam in any suitable manner. For example, xy-scanner 26 may include a pair of galvanometrically-actuated scanner mirrors that can be tilted about mutually perpendicular axes. As another example, xy-scanner 26 may include an electro-optical crystal that can electro-optically steer the beam or an acousto-optical crystal that can acousto-optically steer the beam. As another example, xy-scanner 26 may include a fast scanner that can create, e.g., a 3D matrix of laser pulses. Examples of such scanners include a galvo scanner, resonant scanner, or acousto optical scanner.

Beamsplitter 28 comprises one or more beamsplitters that couple imaging system 22 and laser device 24 into the optical paths towards and/or from eye 12. An example of beamsplitter 28 includes a dichroic mirror. Controller 30 instructs the components of system 10 to performs operations. For example, controller 30 may align the scanning by the DMD device and the scanning by xy-scanner 26 by performing real-time synchronization and position compensation of the scanning. Controller 30 may align the scanning by monitoring the scans with an image sensor, e.g., a CMOS image sensor.

FIG. 2 illustrates an example of a DMD confocal microscope 20 that may be used in system 10 of FIG. 1. In the example, DMD confocal microscope 20 includes a light source 30, a lens 32, and mirror 34, a lens 36, a DMD device 40, relay optics 41 (including, e.g., a lens 42, beamsplitter 46, lenses 48 and 50), a lens 52, and an image sensor 54, optically coupled as shown.

As an overview of operation, light source 30 provides a microscope imaging beam. DMD device 40 directs the microscope imaging beam 43 along an imaging path through relay optics 41 towards the eye and receives the microscope imaging beam reflected from the eye through relay optics 41. DMD device 40 rejects light of the reflected microscope imaging beam that is not from an image plane to scan the microscope imaging beam to yield beam 45. That is, DMD device 40 operates as a SLO pinhole, creating a voxel that rejects light not from the image plane (out of object plane light). Mirror 34 directs beam 45 to image sensor 54. Image sensor 54 detects the scanned the microscope imaging beam to generate the images of the eye.

Turning to the components, light source 30 directs light onto DMD device 40 to illuminate DMD device 40. Light source 30 may provide any suitable light, e.g., broadband white or coherent light.

DMD device 40 comprises an array of micromirrors of any suitable size, e.g., approximately 5×5 um. Individual micromirrors can be toggled to an off or on state (not activated or activated, respectively) to deflect light. When a mirror is activated, it deflects lights towards image sensor 54. Accordingly, DMD device 40 operates like an SLO pinhole, creating a voxel that rejects light not from the image plane (out of object plane light). By toggling the micromirrors in a specific pattern, e.g., by toggling on a set of micromirror(s) that operate as one or more pinhole(s) to scan the microscope imaging beam, DMD device 40 scans an object (e.g., the retina) to acquire an image for image sensor 54.

The pattern of micromirrors may create one or more pinholes. Multiple pinholes allow for acquisition of multiple image points, yielding faster scanning. In general, imaging biological tissue is highly scattering, so the scanned image points should sufficiently far apart from each other to avoid points interfering with adjacent points.

In certain embodiments, DMD device 40 scans an xy-plane to generate a 2D enface image at a z-location. A 2D enface image may be used to track a floater. To generate a 3D image, microscope 20 generates a 2D enface image at an xy-plane at a z-location. Microscope 20 then adjusts the focus to a next z-location to generate the next 2D enface image at the next xy-plane. Controller 30 combines the 2D images to generate the 3D image.

Relay optics 41 comprises one or more optical elements that transport light between system 10 and eye 12. In general, an optical element can act on (e.g., transmit, reflect, refract, diffract, collimate, condition, shape, focus, modulate, and/or otherwise act on) a light beam. Examples of optical elements include a lens, prism, mirror, diffractive optical element (DOE), holographic optical element (HOE), and spatial light modulator (SLM).

In the example, relay optics 41 receive light from DMD device 40 and direct the light to eye 12. Relay optics 41 receive light reflected from eye 12 and direct the light to DMD device 40. A lens of relay optics 41 may be adjusted to change the z-location of the focal position the beam. This allows for multiple 2D enface images to be acquired at different z-locations, which may be used to generate a 3D image. In addition, the afocal relay may be used to image the DMD onto the retina of the of the eye. The optical relay can be designed such that the pupil projected by lens 42 is conjugate to the pupil of the eye, so while the optical beam is traversing the retina, the movement of the beam at the pupil of the eye is minimized to reduce or eliminate vignetting. Also, adjusting the lenses in the afocal relay (lens distance between 48 and 50) can accommodate for patient refractive error.

Mirror 34 comprises any suitable optical element that can direct imaging beam 45 to image sensor 54, such as a mirror or beamsplitter. In certain embodiments, mirror 34 implements darkfield functions to enhance the floater image. In the embodiments, mirror 34 comprises a pickoff mirror that blocks the central cone of the light 43 but allows the outer cone to pass. Light 45 from DMD device 40 is reflected by 34 towards image sensor 54. Image sensor 54 detects light deflected from DMD device 40. Image sensor 54 may comprise any suitable detector, such as a CMOS image sensor.

Certain embodiments of the surgical systems may provide advantages. For example, DMD device 40 scans quickly to generate multiple images in parallel, which allows for fast generation of 2D and 3D images of the eye. As another example, the double-pass DMD reduces stray light reflected by the retina, which improves image contrast when imaging floaters. In addition, the OCT device of imaging system 22 provides enhance depth resolution. As another example, DMD chips are less expensive than the SLO or OCT scanners needed for reasonable image acquisition speeds.

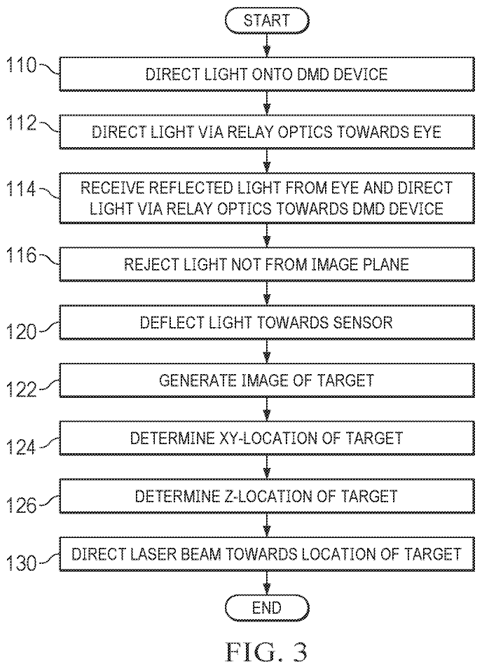

FIG. 3 illustrates an example of a method for imaging and treating an eye that may be performed by an ophthalmic surgical system, such as system 10 of FIG. 1, according to certain embodiments. The system may include a DMD confocal microscope (which includes a DMD device, relay optics, and an image sensor), an imaging system (which includes an OCT device), and a controller.

The method starts at step 110, where a light source directs a light beam onto a DMD device. The DMD device directs the light beam through relay optics to the eye at step 112. The eye reflects the light beam. The DMD device receives the reflected light beam from the eye through the relay optics at step 114.

The DMD device rejects light not from the image plane at step 116 in order to scan the light beam. For example, the DMD toggles micromirrors on and off in a pattern of one or more pinholes to scan the light beam. The DMD device deflects light towards the image sensor at step 120, which generates a sensor signal in response to detecting the light.

The controller generates images of target at step 122 from the signal from the image sensor. For example, the controller may generate a 2D image and/or may generate a 3D image from a plurality of 2D images. The controller determines the xy-location of target at step 124 and the z-location of target at step 126. For example, the controller may determine the xy-location from the DMD device and may determine the z-location from the OCT device. A laser device directs a laser beam towards the location of the target at step 130.

A component (such as a computer or controller described herein) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. An ophthalmic laser surgical system for imaging and treating a target in an eye, comprising:
a digital micromirror device (DMD) confocal microscope configured to generate a plurality of images of the eye, an axis of the eye defining a z-axis, the z-axis defining a plurality of xy-planes, the DMD confocal microscope comprising:
a light source configured to provide a microscope imaging beam;
a DMD device comprising an array of micromirrors configured to:
direct the microscope imaging beam along an imaging path towards the eye;
receive the microscope imaging beam reflected from the eye; and
reject light of the microscope imaging beam reflected from the eye that is not from an image plane to scan the microscope imaging beam; and
an image sensor configured to detect the scanned microscope imaging beam to generate the plurality of images of the eye;
a laser device configured to direct a laser beam along a laser beam path towards the target in the eye; and
a computer configured to send instructions to the DMD confocal microscope and the laser device.

2. The ophthalmic laser surgical system of claim 1, the target comprising a vitreous eye floater.

3. The ophthalmic laser surgical system of claim 1, the DMD device configured to reject light of the microscope imaging beam reflected from the eye that is not from the image plane to scan the microscope imaging beam by:
toggling on a set of one or more micromirrors that operate as a pinhole to scan the microscope imaging beam.

4. The ophthalmic laser surgical system of claim 1, the DMD device configured to reject light of the microscope imaging beam reflected from the eye that is not from the image plane to scan the microscope imaging beam by:
toggling on a set of one or more micromirrors that operate as a plurality of pinholes to scan the microscope imaging beam.

5. The ophthalmic laser surgical system of claim 1, the DMD confocal microscope configured to:
generate a plurality of two-dimensional (2D) enface images at the plurality of xy-planes.

6. The ophthalmic laser surgical system of claim 5, the DMD confocal microscope configured to:
combine the plurality of two-dimensional (2D) enface images to generate a three-dimensional (3D) image.

7. The ophthalmic laser surgical system of claim 1, further comprising an imaging system configured to generate a second plurality of images of the eye.

8. The ophthalmic laser surgical system of claim 7, the imaging system comprising an optical coherence tomography (OCT) device configured to generate at least one of the second plurality of images of the eye.

9. The ophthalmic laser surgical system of claim 7, the imaging system comprising a scanning laser ophthalmoscope (SLO) device configured to generate at least one of the second plurality of images of the eye.

10. The ophthalmic laser surgical system of claim 7, the imaging system configured to determine a z-location of the target relative to the z-axis.

11. The ophthalmic laser surgical system of claim 7, further comprising an xy-scanner configured to:
receive an imaging beam from the imaging system and direct the imaging beam along an imaging system beam path towards the eye; and
receive the laser beam from the laser device and direct the laser beam along the laser beam path aligned with the imaging system beam path towards the eye.

12. A method for imaging and treating a target in an eye, comprising:
generating, by a digital micromirror device (DMD) confocal microscope, a plurality of images of the eye, an axis of the eye defining a z-axis, the z-axis defining a plurality of xy-planes, the generating comprising:
providing, by a light source of the DMD confocal microscope, a microscope imaging beam;
directing, by an array of micromirrors of a DMD device of the DMD confocal microscope, the microscope imaging beam along an imaging path towards the eye;
receiving, by the array of micromirrors, the microscope imaging beam reflected from the eye;
rejecting, by the array of micromirrors, light of the microscope imaging beam reflected from the eye that is not from an image plane to scan the microscope imaging beam; and
detecting, by an image sensor of the DMD confocal microscope, the scanned microscope imaging beam to generate the plurality of images of the eye; and
directing, by a laser device, a laser beam along a laser beam path towards the target in the eye.

13. The method of claim 12, the target comprising a vitreous eye floater.

14. The method of claim 12, the rejecting light of the microscope imaging beam reflected from the eye that is not from the image plane to scan the microscope imaging beam comprising:
toggling on a set of one or more micromirrors that operate as a pinhole to scan the microscope imaging beam.

15. The method of claim 12, the rejecting light of the microscope imaging beam reflected from the eye that is not from the image plane to scan the microscope imaging beam comprising:
toggling on a set of one or more micromirrors that operate as a plurality of pinholes to scan the microscope imaging beam.

16. The method of claim 12, further comprising:
generating, by the DMD confocal microscope, a plurality of two-dimensional (2D) enface images at the plurality of xy-planes.

17. The method of claim 16, further comprising:
combining, by the DMD confocal microscope, the plurality of two-dimensional (2D) enface images to generate a three-dimensional (3D) image.

18. The method of claim 12, further comprising:
generating, by an imaging system, a second plurality of images of the eye.

19. The method of claim 18, further comprising:
generating, by an optical coherence tomography (OCT) device of the imaging system, at least one of the second plurality of images of the eye.

20. The method of claim 18, further comprising:
generating, by a scanning laser ophthalmoscope (SLO) device of the imaging system, at least one of the second plurality of images of the eye.

21. The method of claim 18, further comprising:
determining, by the imaging system, a z-location of the target relative to the z-axis.

22. The method of claim 18, further comprising:
receiving, by an xy-scanner, an imaging beam from the imaging system and directing the imaging beam along an imaging system beam path towards the eye; and
receiving, by the xy-scanner, the laser beam from the laser device and directing the laser beam along the laser beam path aligned with the imaging system beam path towards the eye.
